(19) United States
(12) Patent Application Publication
(10) Pub. No.: US 2020/0138619 A1
(43) Pub. Date: May 7, 2020

(54) PERISTOMAL SUPPORT BELT

(71) Applicant: Hollister Incorporated, Libertyville, IL (US)

(72) Inventors: George J. Cisko, Jr., Spring Grove, IL (US); Mark W. Jockel, Chicago, IL (US); Christina Augustyn, Chicago, IL (US); Lynn Sacramento, Libertyville, IL (US); Xuemei Wang, Buffalo Grove, IL (US); Larisa Mats, Buffalo Grove, IL (US); Paul C. Fletter, Mt. Prospect, IL (US); Ryan S. Park, Northbrook, IL (US)

(21) Appl. No.: 16/303,998

(22) PCT Filed: Jun. 2, 2017
(86) PCT No.: PCT/US2017/035648

§ 371 Date: Nov. 21, 2018

Related U.S. Application Data
(60) Provisional application No. 62/352,261, filed on Jun. 20, 2016.

(51) Int. Cl. A61F 5/449

(52) U.S. Cl. CPC A61F 5/449

(57) ABSTRACT

A peristomal support belt system includes a supply of material having a first length and a first end and a buckle configured for attachment to the first end. The supply of material is cut to a second length to form a section of material having the first end and a second end, the second length extending from the first end to the second end. The buckle is configured for attachment to the second end. A method of making a peristomal support belt and a peristomal support belt are also provided.

### BACKGROUND

The following description generally relates to ostomy appliances or accessories, and in particular, a peristomal support belt that is customizable by a patient or caretaker for use on a patient.

Ostomy pouches for collecting bodily waste are used by patients who have had surgery such as a colostomy, ileostomy, or urostomy. Ostomy pouches can be connected to the patient in a variety of ways. In one arrangement, a faceplate includes a body-facing side, a pouch-facing side, an opening and a faceplate coupling section. The body-facing side is adhered to the patient at a peristomal area. The opening is configured to receive the stoma therethrough. The coupling section is formed on the pouch-facing side, typically near or around the opening. The ostomy pouch includes an inlet and a pouch coupling section near or around the inlet. The pouch coupling section is coupled to the faceplate coupling section such that the opening and the pouch inlet are aligned. In other configurations, the faceplate and ostomy pouch may be integrally formed as a one-piece system. In both configurations, bodily waste may be received from the stoma into the pouch.

Some arrangements for connecting an ostomy pouch to a patient also include an ostomy belt to provide supplemental support to an adhesive barrier. Typically, the ostomy belt extends almost completely around the patient's waist or abdominal region and has the ostomy pouch secured thereto by way of belt tabs formed on the pouch. However, this system may not provide adequate abdominal support, especially around hernias. Further, the patient may find this system visually displeasing as abdominal bulges or shapes of ostomy appliances and accessories may be visible. Another ostomy belt may be formed having a greater height to provide support to the patient's abdominal region.

However, in the ostomy belts above, adequate peristomal support may not be provided due, in part, to an opening at the coupling portion of belt. Thus, adequate support may not be provided where a parastomal hernia exists, or, alternatively, to prevent a parastomal hernia from forming.

Hernia support belts have been developed to provide support to and/or reduce the risk of parastomal hernias. Hernia support belts are typically used in combination with pouch coupling assemblies where an ostomy faceplate is adhered to a patient's skin in the peristomal area and the pouch is coupled to the faceplate. The hernia support belt extends about the patient's abdomen and applies pressure over the ostomy pouch and faceplate, thereby providing peristomal support. However, the hernia support belts may limit or prevent access to the ostomy pouch by extending over the ostomy pouch. As such, to replace an ostomy pouch, the patient or caretaker typically needs to remove the hernia support belt, decouple the pouch from the faceplate, reattach an empty or cleaned pouch to the faceplate, and refit the hernia support belt around the patient. This process may be time consuming and allow for a period of time where the support is not provided to the parastomal area.

Another hernia support belt extends around the patient's abdomen and over a faceplate adhered to the abdomen. The belt includes an opening substantially aligned with the faceplate. The pouch may extend through the opening for attachment to the faceplate. In use, the belt may apply pressure to a faceplate assembly.

The ostomy and hernia support belts above are manufactured in a variety of different, standard sizes to fit patients of different sizes. The ostomy and hernia support belts are also manufactured to include various features such as buckles and openings for ostomy appliance attachment. On the manufacturing end, these processes may be time consuming, labor intensive and costly. In addition, the standard sizes may not properly fit all patients. An ill-fitting ostomy belt may cause discomfort to the patient, adversely affect sealing capabilities of the ostomy appliance around the stoma, and/or be visually displeasing. Further, an opening may not be accurately positioned around the peristomal area. Further still, an ill fitted ostomy or hernia support belt may not apply adequate peristomal pressure area to support or prevent hernias.

Some ostomy and hernia support belts may be manufactured in custom sizes for specific patients. However, this customization increases manufacturing time, and in turn, delivery time to the patient. Thus, to maintain an adequate supply of ostomy or hernia support belts, the patient or caretaker must account for the manufacturer's extended turnaround time when re-ordering a supply. Further, it may be difficult for the patient or caretaker to timely obtain a custom fitted ostomy or hernia support belt when there is an immediate or urgent need to acquire a new belt.

Accordingly, it is desirable to provide a peristomal support belt that is easily customizable and/or sized by an end user patient or caretaker and capable of adequately supporting and/or preventing parastomal and other hernias. In addition, it is desirable to provide a modular peristomal support belt that may be manufactured or assembled by the end user patient or caretaker in the customized size from a common supply or kit.

### SUMMARY

According to one embodiment, there is provided a peristomal support belt system having a supply of material having a first length and a first end, and a buckle configured for attachment to the first end. The supply of material is cut to a second length to form a section of material having the first end and a second end, the second length extending from the first end to the second end. The buckle is configured for attachment to the second end.

According to another embodiment there is provided a method of making a peristomal support belt. The method includes providing a supply of material, cutting the supply of material to form a first section of material having a first end, a second end and a first length extending from the first end to the second end, attaching a buckle to one of the first end and the second end of material and attaching the buckle to the other of the first end and the second end of material.

According to still another embodiment, there is provided a peristomal support belt including a section of material having a first end, a second end and a length extending from the first end to the second end, a buckle removably fastened to the first end and the second end, an opening formed in the section of material and a grommet disposed in the opening.

Other objects, features, and advantages of the disclosure will be apparent from the following description, taken in conjunction with the accompanying sheets of drawings, wherein like numerals refer to like parts, elements, components, steps, and processes.

### BRIEF DESCRIPTION OF THE DRAWINGS

FIG. 1 is a perspective view of a peristomal support belt system according to an embodiment described herein; and

FIG. 2 shows a peristomal support belt according to an embodiment described herein.

### DETAILED DESCRIPTION

While the present disclosure is susceptible of embodiment in various forms, there is shown in the drawings and will hereinafter be described one or more embodiments with the understanding that the present disclosure is to be considered illustrative only and is not intended to limit the disclosure to any specific embodiment described or illustrated.

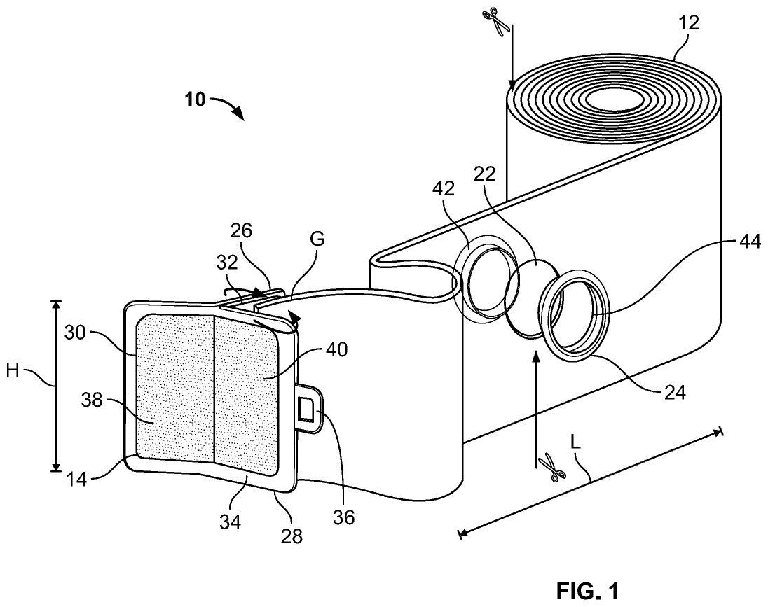

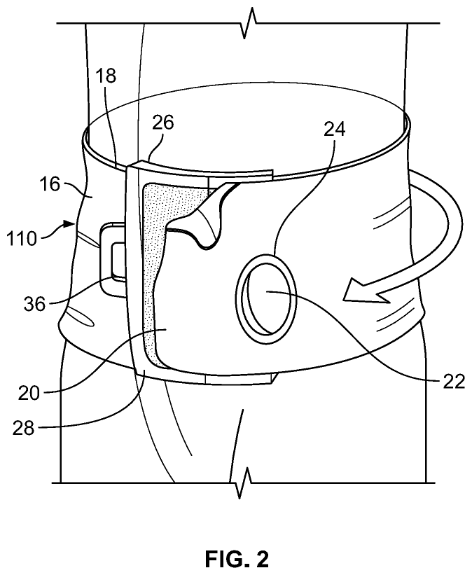

FIG. 1 shows, generally, a peristomal support belt system 10 according to an embodiment described herein, and FIG. 2 shows an example of a peristomal support belt 110 described herein positioned on a patient. Referring to FIG. 1, the peristomal support belt system 10 generally includes a supply of material 12 and a buckle 14. The supply of material 12 has a first, initial length. In one embodiment, the supply 12 may be a roll of material.

The material is preferably a breathable material, such as a cloth or fabric. The material may be woven or non-woven. In one embodiment, the material is suitable for fastening by way of a hook and loop fastener, such as VELCRO®, to the buckle 14. The material may have a hook and loop fastener section positioned or secured thereon, or the material itself may be suitable for serving as a portion of the hook and loop fastener. That is, the material may, for example, be formed to, or inherently include, a plurality of loops suitable for engagement with a plurality of hooks to attach to the material 12 to the buckle 14, or vice versa. In one embodiment, the material 12 may include an elastic section or sections, or elastic strands, to provide the material with elasticity in at least a length direction L.

The material supply 12, according to one embodiment, may be cut by an end user to a second length to form a first section of material 16 (FIG. 2). The end user may be, for example, a patient or a caretaker. The first section of material 16 includes a first end 18 and a second end 20 (see FIG. 2). The second length extends from the first end 18 to the second end 20. The second length of material, i.e., the length of the first section 16, is generally customized to fit around an abdominal or waist area of the patient as a peristomal support belt 110 (see FIG. 2). That is, the size of the peristomal support belt (e.g., the length) 110 may be customized by the end user to fit a specific patient. In one embodiment, the first end 18 may overlap the second end 20 when positioned on a patient. However, in other embodiments, the first and second ends 18, 20 may be positioned generally adjacent to one another, or spaced from one another when disposed on the patient. A single supply of material 12 may be formed into a plurality of peristomal support belts 110 having the same or different sizes configured to fit patients of different sizes.

The first section 16 also has a height H, extending in a direction transverse to the length. The height H of the first section 16 is configured to extend in a height direction along the patient's abdominal area. Accordingly, the first section 16, when formed as the peristomal support belt 110 as shown in FIG. 2, may provide support to the patient's abdominal area. For example, support may be provided to hernias, including parastomal hernias. Alternatively, or in addition, support provided by the peristomal support belt may assist in the prevention of hernias, including parastomal hernias.

The first section 16 may include an opening 22 formed therein. In one embodiment, the opening 22 is formed by cutting away a portion of the material. However, it is understood that the opening 22 may be formed using other techniques as well. For example, the opening 22 may be formed by punching or other similar techniques. The opening 22 may also be formed by removing a weakened section of material, for example, a perforated section.

A grommet 24 may be positioned in the opening 22. The grommet 24 may reinforce a perimeter of the opening 22 to limit unintentional tearing of the opening and limit or prevent interference from the perimeter opening when coupling an ostomy pouch (not shown) to an underlying faceplate assembly (not shown) on the patient in an area of the opening 22. Further, the grommet 24 may apply a localized pressure to the faceplate or directly to the patient in the peristomal area, which may support or prevent a parastomal hernia. In one embodiment, the grommet 24 may be formed as a substantially circular or ring shaped piece. However, other shapes are envisioned as well. For example, the grommet 24 may be substantially plate-shaped so as to overlap a larger portion of the first section 16 than the ring shaped piece shown in the example of FIG. 1. The plate-shaped grommet 24 includes an opening corresponding to the opening 22 in the first section 16. The plate-shaped grommet 24 may be urged against the faceplate or the patient over a larger surface area, and thus may provide peristomal support over a larger area, compared to the ring-shaped example.

In one embodiment, the first section 16 has a generally constant height H along its length. However, the present disclosure is not limited to this configuration. For example, in another embodiment, the first section 16 may have a varying height H. In one configuration, the first section 16 may have a section of increased height H along a portion of its length, for example, in the region of the opening 22. The increased height H may be formed along a portion of the length beginning at a position on one side of the opening 22 in the length direction L and extending to a position on another side of the opening 22 in the length direction L. The increased height H section may be formed along one of, or both, a top edge and bottom edge of the first section 16.

In one embodiment, the increased height H section may be formed as a gradual, sloping transition over a portion of the length of the first section 16, such that the height H of the belt gradually, over a length, increases to a maximum height H, and gradually, over another length, decreases to a lower height H. In one embodiment, the height H may be constant over at least a portion of the increased height H section. Alternatively, or in combination, the increased height H section may be formed with one or more stepwise increases or decreases in height. The supply of material 12 may be formed having a plurality of sections of increased height so that multiple first sections 16, each corresponding to an individual peristomal support belt, may be formed from a single supply 12.

The peristomal support belt system 10 also includes the buckle 14. The buckle 14 is configured to secure the first end 18 and second end 20 together when the first section of material 16 is fitted around the patient as a peristomal support belt. In one embodiment, the buckle 14 includes a first arm 26, a second arm 28 and, optionally, a body 30. The first arm 26 and the second arm 28 may be joined to, or formed integrally with, the body 30 and extend away from one another forming a gap G therebetween. At least one of the first and second arms 26, 28 is preferably formed from a flexible material such that at least one of arms 26, 28 may be moved toward and away from the other. Each arm 26, 28 includes an interior surface 32 and an exterior surface 34. The interior surfaces 32 are disposed on opposite sides of the gap G and generally face one another. In one embodiment, at least one of the first and second arms 26, 28 includes a tab 36 configured for grasping by a user to manipulate the first and/or second arms 26, 28. Further, in one embodiment, each arm 26, 28 may be formed as a substantially planar section. However, the present disclosure is not limited to this configuration, and curved or non-planar sections are envisioned as well.

The body 30 extends from the first and second arms 26, 28 and includes exterior surfaces 38 on opposite sides thereof. Accordingly, in one embodiment, the buckle 14 may be formed substantially in a “Y” shape. Further, in one embodiment, the body 30 is formed as a substantially planar section. However, differently shaped sections are also envisioned. For example, the body 30 may be curved so as to substantially match a contour of the patient's body. In another embodiment, the body 30 may be omitted, such that the buckle 14 is formed substantially in a “V” shape by the first and second arms 26, 28.

The buckle 14 includes a fastener 40 configured for securing the buckle 14 to the material 16. In one embodiment, the first end 18 of the material is received between the first arm 26 and the second arm 28 and secured thereto, and the second end 20 of material 16 is secured to at least one of the body 30 and exterior surfaces 34 of the first or second arms 26, 28. In one embodiment, the fastener 40 extends on one or more of the exterior surfaces 38 of the body 30, one or more of the exterior surfaces 34 of the first and second arms 26, 28, and one or more of the interior surfaces 32 of the first and second arms 26, 28. In one embodiment, the buckle 14 may be formed having substantially the same height H as the first section of material 16. However, it is understood that the height of the buckle 14 is not limited to this example.

In one embodiment, the fastener 40 may be a hook-and-loop fastener such as VELCRO®. A portion of the fastener 40 is disposed on the buckle 14 as described above, and a corresponding or mating portion of the fastener 40 is disposed on the supply of material 12 (and in turn, on the first section of material 16), or formed by the material itself. Preferably, the fastener 40 is a releasable fastener so that the buckle 14 can be attached to and removed from the first material 16 as desired. Although hook-and-loop fasteners are described in the example above, it is understood that other releasable fasteners are suitable as well, including, but not limited to, buttons, snaps, hooks, adhesives, pins, straps, slide fasteners and the like. It is also understood that one or more different types of fasteners may be implemented on a buckle 14. For example, the buckle 14 may include a first fastener of a first type to attach to the first end 18 of the material 16, and a second fastener 20 of a second type to attach to the second end of the material 16. Accordingly, the buckle 14 may be re-used with other peristomal support belts. It is understood that the present disclosure is not limited to a single fastener or single type of fastener, however.

A method of making the peristomal support belt 110, according to an embodiment described herein, includes providing the supply of material 12 and cutting the supply of material 12 to form the first section of material 16. The first section of material 16 includes a first end 18 and a second end 20, and has a first length extending from the first end 18 to the second end 20. The method also includes attaching the buckle 14 to one of the first end 18 and the second end 20, and attaching to the buckle 14 to the other of the first end 18 and the second end 20.

In one embodiment, attaching the buckle 14 to one of the first end 18 and the second end 20 includes positioning the first end 18 between the first and second arms 26, 28. Attaching the buckle 14 to the other of the first end 18 and the second end 20 includes attaching the body 30 to the second end 20. The method may further include forming the opening 22 in the first section of material 16. The grommet 24 may be disposed in the opening 22 and receive a portion of an ostomy appliance, such as an ostomy pouch, therethrough for connection to a faceplate assembly adhered to the patient.

The method may further include cutting the supply 12 of material to form second, and subsequent, sections of material, each section having a first end and a second end and a length extending from the first end to the second end. The method described above with respect to the first section 16 may then be carried out on the second and subsequent sections as well, to form a plurality of peristomal support belts. The buckle 14 may be reused. For example, the buckle 14 may be removed from the ends 18, 20 of the first section of material 16 and be attached to the ends of the second and/or subsequent sections of material.

In the embodiments above, each section of material 16 cut from the supply of material 12 may be used to form a peristomal support belt 110. As shown in FIG. 2, the peristomal support belt 110, according to one embodiment, includes the section of material 16 having a first end 18 and a second end 20, and a length extending from the first end 18 to the second end 20. The buckle 14 is removably fastened to the first end 18 and the second end 20, and an opening 22 may be formed in the section of material. In use, the opening 22 generally corresponds to the stomal area on the patient. The grommet 24 is disposed in the opening 22 to reinforce the opening 22. A portion of an ostomy appliance, such as an ostomy pouch, may extend through opening for connection to a corresponding ostomy faceplate adhered to the patient. In one embodiment, the grommet 24 may be a two-part grommet having an inner part 42 and an outer part 44 secured to one another. The size of opening 22 and the grommet 24, 42 may be provided in various sizes to accommodate different size ostomy pouches and flanges.

Accordingly, in the embodiments above, a peristomal support belt 110 may be assembled with dimensions customized to fit an end user rather than in standard sizes. Thus, a patient wearing the peristomal support belt may experience improved fit and comfort compared to peristomal support belts supplied in standard sizes. In addition, an end user, such as the patient or caretaker may quickly and easily assemble each peristomal support belt 110 from the supply of material 12 and the buckle 14. Thus, manufacturing time and costs at a manufacturing or supplier facility may be reduced. That is, the peristomal support belts 110 may be assembled on demand and immediately used by a patient, without having to wait for manufacturer assembly and shipping time.

In addition, a pressure applied by the section of material 16 in the peristomal area may be supplemented by the grommet 24. For example, the grommet 24 may apply a localized pressure to the peristomal area providing increased peristomal support. Accordingly, the peristomal support belt of the embodiments described herein may apply pressure to a peristomal area to either support, assist in the prevention of, or prevent parastomal hernias.

It is understood that the features described with respect to any of the embodiments above may be implemented, used together with, or replace features described in any of the other embodiments above.

All patents referred to herein, are hereby incorporated herein in their entirety, by reference, whether or not specifically indicated as such within the text of this disclosure.

In the present disclosure, the words “a” or “an” are to be taken to include both the singular and the plural. Conversely, any reference to plural items shall, where appropriate, include the singular.

From the foregoing it will be observed that numerous modifications and variations can be effectuated without departing from the true spirit and scope of the novel concepts of the present invention. It is to be understood that no limitation with respect to the specific embodiments illustrated is intended or should be inferred. The disclosure is intended to cover by the appended claims all such modifications as fall within the scope of the claims.

1. A peristomal support belt system comprising:
a supply of material having a first length and a first end; and
a buckle configured for attachment to the first end,
wherein the supply of material is cut to a second length to form a section of material having the first end and a second end, the second length extending from the first end to the second end, and
wherein the buckle is configured for attachment to the second end.

2. The peristomal support belt system of claim 1, wherein the material is a breathable fabric.

3. The peristomal support belt system of claim 1, wherein the material includes an opening.

4. The peristomal support belt system of claim 3, further comprising a grommet disposed at the opening.

5. The peristomal support belt system of claim 1, wherein the buckle is attached to the material with a hook and loop fastener.

6. The peristomal support belt system of claim 1, wherein the buckle includes first and second arms configured to receive the first end therebetween.

7. The peristomal support belt system of claim 6, wherein the buckle includes a body portion extending from the first and second arms configured for attachment to the second end.

8. The peristomal support belt system of claim 1, wherein the supply of material is a roll of material.

9. A method of making a peristomal support belt comprising:
providing a supply of material;
cutting the supply of material to form a first section of material having a first end, a second end and a first length extending from the first end to the second end;
attaching a buckle to one of the first end and the second end of material; and
attaching the buckle to the other of the first end and the second end of material.

10. The method of claim 9, wherein the buckle includes first and second arms and attaching the buckle to one of the first end and the second end includes positioning the first end between the first and second arms.

11. The method of claim 10, wherein the buckle further includes a body extending from the first and second arms, and attaching the buckle to the other of the first end and the second end includes attaching the body to the second end.

12. The method of claim 9, further comprising forming an opening on the first section of material.

13. The method of claim 12, further comprising positioning a grommet at the opening.

14. The method of claim 9, further comprising cutting the supply of material to form a second section of material having, a third end, a fourth end and a second length extending from the third end to the fourth end.

15. A peristomal support belt comprising:
a section of material having a first end, a second end and a length extending from the first end to the second end;
a buckle removably fastened to the first end and the second end;
an opening formed in the section of material; and
a grommet disposed in the opening.

16. The peristomal support belt of claim 15, wherein the material is a breathable material.

17. The peristomal support belt of claim 15, wherein the buckle includes first and second arms, and the first end is received between the first and second arms to be fastened to the buckle.

18. The peristomal support belt of claim 17, wherein the buckle includes a body extending from the first and second arms and the second end is fastened to the body.

19. The peristomal support belt of claim 18, wherein at least one of the first and second arms includes a tab configured to the manipulated by a user to move the first and/or second arms.

20. The peristomal support belt of claim 18, wherein the buckle includes a fastener on one or more exterior surfaces of the body portion and on one or more interior surfaces of the first and second arms, the wherein the fastener is a hook and loop fastener.
